(19) United States
(12) Patent Application Publication
(10) Pub. No.: US 2006/0292030 A1
(43) Pub. Date: Dec. 28, 2006

(54) Process for the preparation of sterile polysaccharide solutions

(76) Inventors: Erich Odermatt, Schaffhausen (CH); Juergen Wegmann, Stockach (DE)

Correspondence Address: NATH & ASSOCIATES, 112 South West Street, Alexandria, VA, 22314, US

(21) Appl. No.: 11/453,250

(22) Filed: Jun. 15, 2006

(30) Foreign Application Priority Data
Jun. 17, 2005 (DE) 102005030011.1

(51) Int. Cl. A01N 43/04

(52) U.S. Cl. 422/13; 514/54; 514/55; 514/56; 514/57; 514/59; 514/60

(57) ABSTRACT

A novel process for the preparation of sterile polysaccharide solutions from at least one packed, aqueous unsterile dispersion of at least one thermolabile and/or poorly soluble polysaccharide is provided, where the dissolution of the polysaccharide and the sterilization are effected simultaneously by a heat treatment at above 100° C. The invention furthermore relates to a sterile solution of at least one thermolabile and/or poorly soluble polysaccharide.

The present invention relates to sterile polysaccharide solutions, and to their preparation.

The preparation of pharmaceutical and medical products based on sterilized polysaccharide solutions is of outstanding importance, in particular for the preparation of liquid and gelatinous haemostyptics, gels for adhesion prophylaxis, and medical tissue adhesives.

Generally, a number of methods are available for the sterilization of solid polysaccharides and polysaccharide mixtures, the sterilization usually being carried out by means of gamma or electron irradiation or by treatment with ethylene oxide. With regard to the sterilization of aqueous polysaccharide solutions, the conventional sterilization methods, however, have some disadvantages. Thus, the sterilized products often do not have the desired properties, for example gelation, at all or only to an unsatisfactory extent. In the case of sterilization by means of energy-rich irradiation, for example gamma or electron irradiation, this is in particular to be attributed to the fact that the water molecules are cleaved into chemically highly reactive hydroxyl radicals, which in turn can cause further free radical reactions in the dissolved polysaccharide chains. Breaks in the chain can result in this manner, which in turn leads to a reduction of the molecular weight or to crosslinking reactions. These are processes which are not controllable, which can adversely affect the properties of the polysaccharides and the function of the products produced therefrom in an undesired manner.

The ethylene oxide sterilization of aqueous polysaccharide solutions has the disadvantage that the toxic ethylene oxide is soluble in water and therefore its removal after the sterilization process is problematical. The same also applies for chemical sterilization processes, in particular using glutaraldehyde or formaldehyde, which are moreover very reactive substances which can enter into undesired secondary reactions with polysaccharides.

Sterile filtration is admittedly a powerful sterilization alternative, but only for dilute aqueous polysaccharide solutions. Highly viscous solutions, however, can only be filtered with a low flow rate, if at all. The filter materials block up very rapidly and must therefore be changed frequently, so this process can entail very high costs.

In addition to the disadvantages just mentioned, in the preparation of sterile aqueous polysaccharide solutions the problem of the dissolution of the corresponding polysaccharides in water before or during the sterilization process is raised, in particular with poorly soluble polysaccharides for the preparation of highly concentrated aqueous sterile polysaccharide solutions. For their preparation, the polysaccharides are dissolved in water, customarily at relatively high temperatures, in particular between 60° C. and 80° C., in some cases over the course of a number of days. The solutions are subsequently slowly cooled to room temperature. In this manner, supersaturated aqueous solutions of the polysaccharides concerned are obtained. However, this process is only suitable for thermostable polysaccharides. Thermolabile polysaccharides, however, can in particular fragment or split off side chains or rearrange as a result of long heat treatment. This leads in the majority of cases to a loss or at least to an undesired adverse effect on the properties of the polysaccharide solutions or the products produced therefrom.

The object is therefore to make available a process for the preparation of aqueous sterile polysaccharide solutions from thermolabile and/or poorly soluble polysaccharides while circumventing the said disadvantages of the prior art. In this process, the physicochemical properties of the polysaccharides or polysaccharide solutions should in particular not be adversely affected in order to guarantee their applicability in medicine and surgery. The process should moreover make the aqueous sterile polysaccharide solutions available in a ready-to-use form, the application of which can thus take place rapidly and safely in a simple manner, in particular in medical and surgical interventions.

This object is achieved by a process for the preparation of sterile polysaccharide solutions, where at least one aqueous unsterile dispersion of at least one thermolabile and/or poorly soluble polysaccharide is packed and the dissolution of the polysaccharide and the sterilization are effected simultaneously by heat treatment of the packing at above 100° C.

Below, the term dispersion in the sense of the invention should be understood as meaning a system of at least two phases, where one phase is continuous and liquid (dispersing agent) and at least one further phase is present in the form of a finely divided solid (dispersed phase). Therefore the term dispersion in the sense of the invention expressly also comprises suspensions, gels and pastes.

A thermolabile polysaccharide should be understood below as meaning a polysaccharide whose properties and/or structure are disadvantageously influenced or altered, in particular in a range of hours, on heat treatment.

A poorly soluble polysaccharide should be understood below as meaning a polysaccharide which is not soluble in water or a water mixture in the concentration desired for the respective application at room temperature or surrounding temperature.

In one particular embodiment of the process according to the invention, two polysaccharides in each case present separately of one another in the form of an aqueous unsterile dispersion, which are preferably able to react with one another, are subjected to heat treatment. Preferably, the polysaccharides, which in each case are present dissolved and sterilized after the heat treatment, can be mixed with one another and applied immediately after or during mixing thereof to the desired application site. Thus a reaction product of the two polysaccharides reacting with one another results at least partially directly at the application site.

Preferably, only one of the two polysaccharides which preferably react with one another is thermolabile and/or poorly soluble and is therefore subjected to a heat treatment for sterilization, while the other polysaccharide is a polysaccharide which is soluble or can be dissolved at room temperature in the desired amounts. According to the preceding embodiments, the solution obtained after heat treatment of the dispersion of the thermolabile and/or poorly soluble polysaccharide and the sterile solution of the soluble polysaccharide can be mixed and applied to the desired application site before or during mixing.

In another embodiment of the process according to the invention, it may be preferable to subject an aqueous unsterile dispersion, which contains two or more polysaccharides, preferably two or three, to a heat treatment. An aqueous sterile solution of two or more, preferably two or three, polysaccharides is thus obtained. Preferably, the polysaccharides are polysaccharides which do not react with one another. Provided a possible reaction product of the polysaccharide mixture does not restrict their applicability, it is also possible to subject an aqueous unsterile dispersion of two or more, preferably two or three, polysaccharides which react with one another to a heat treatment.

Preferably, for the preparation of the sterile polysaccharide solutions at least one modified polysaccharide is used which preferably has lipophilic modifications. Such modifications can be performed, for example, by the introduction of lipophilic substituents, in particular of a sulphydryl group or methyl group. Additionally or as an alternative thereto, functionalities already present in the polysaccharide, in particular carboxyl, aldehyde, alcohol and/or amino groups, can also be converted into lipophilic substituents by suitable chemical reactions, for example esterifications, amidations and/or oxidations. Preferably, at least one aldehyde- and/or amino group-bearing polysaccharide is used.

With advantage, at least one polysaccharide from the group consisting of hyaluronic acid, heparin, chitin, chitosan, alginate, cellulose, starch, amylose, amylopectin, dextran and its derivatives, of which at least one is poorly soluble, is used.

According to the invention, it is particularly preferred that dextran aldehyde is used for the preparation of the sterile polysaccharide solution. A sterile dextran aldehyde solution in particular prepared by the process according to the invention can be mixed with a likewise sterile chitosan solution, in particular sterilized by the process according to the invention. Preferably, a dextran aldehyde solution and a chitosan solution, in particular a sterile 4% strength chitosan solution, are mixed. The gel obtained after mixing is preferably used as a surgical adhesive for tissue closure and in particular for haemostasis.

Preferably, the aqueous unsterile dispersion is prepared by mixing water and polysaccharide. The dispersion is preferably prepared by introduction of the polysaccharide into water. The preparation of the dispersion is carried out in a suitable unit, preferably a homogenizer, which is charged with a specified amount of water, the water in particular being prepared, preferably completely deionized. With advantage, the water has wfi (water for injection) quality. In this manner, it is ensured that the sterilization material is not adversely affected in any manner by constituents dissolved in the water, for example salts and metals.

Alternatively to this, the aqueous unsterile dispersion can also be prepared from a water/DMSO mixture, in particular by introduction of the polysaccharide into such a mixture. Preferably, water/DMSO mixture are used in a ratio of 99.5:0.5% by volume to 50:50% % by volume, in particular of 99.5:0.5% by volume to 90:10% by volume. With regard to further features of the water/DMSO mixtures, reference is made to the above description.

According to the invention, the introduction of the polysaccharide into water or into a water/DMSO mixture can be performed during a time of between 60 min and 120 min, in particular between 80 min and 100 min, preferably during about 90 min. Advantageously, the preparation of the dispersion, in particular the introduction of the polysaccharide into water, is carried out at a temperature between 4° C. and 25° C., in particular between 4° C. and 15° C., preferably at about 6° C.

In a further embodiment of the process according to the invention, lyophilized polysaccharide, for example lyophilized dextran aldehyde, is used for the preparation of the aqueous dispersion.

With advantage, the aqueous dispersion, in particular obtained by the previously described steps of the process according to the invention, is homogenized. This can preferably be carried out after addition of the polysaccharide to the water introduced and preferably prepared, in particular deionized, in the already mentioned unit, in particular in the homogenizer, within a period of time of, for example, about 30 min.

The aqueous dispersion is preferably prepared having a content of polysaccharide of 5 to 20% by weight, in particular 5 to 15% by weight, preferably of about 10% by weight.

The process according to the invention is preferably furthermore distinguished in that the aqueous dispersion is packed in air-tight and in particular air-free form. This is particularly advantageous for the sterilization result, since reproducible temperature/pressure conditions are produced in this way.

In a particularly preferred embodiment, the dispersion of the aqueous polysaccharide solution is transferred to at least one closed container, preferably to a one- or two-chamber syringe. A two-chamber or twin syringe is particularly advantageous as a closed container, since in this way two different aqueous polysaccharide dispersions can simultaneously be separately sterilized and can in particular be applied as a mixture to the desired intended site during a surgical intervention by the normal use of the syringe. Preferably, one of the aqueous dispersions to be sterilized separately is present in the form of a solution. Preferably, one chamber of the two-chamber syringe contains the dispersion of an aldehyde group-bearing polysaccharide, in particular dextran aldehyde, and the other chamber of the two-chamber syringe contains the solution of an amino group-bearing polysaccharide, in particular chitosan. Particularly preferably, one chamber contains a 4% strength chitosan solution. The sterile polysaccharide solutions present after the heat treatment, in particular a sterile dextran aldehyde and a sterile chitosan solution, for example a 4% strength chitosan solution, can, as already mentioned, be mixed to give a surgical tissue adhesive having, in particular, haemostatic action.

In a particularly preferred embodiment of the process according to the invention, the aqueous dispersion, in particular prepared by the previously mentioned steps, is sterilized in the form of a paste or of a gel, where the dispersion can first be present as a suspension, which is converted before sterilization by allowing to stand for a relatively long time, for example within 2 to 3 hours, to a paste or a gel. According to the invention, however, it can likewise be intended that the suspension is subjected immediately to a heat treatment and thus to sterilization.

In a particularly advantageous manner, in the present process according to the invention the dissolution of the polysaccharide and the sterilization are brought about simultaneously by treatment of the packed aqueous dispersion with a vaporizable medium, which under elevated pressure in particular effortlessly achieves the standardized sterilization temperatures, in particular of 121° C. or 134° C. On the one hand, the evaporated medium acts as a heat exchanger on the water of the packed dispersion and on the other hand as a pressure equalization. By the condensation of the water vapour on the cooler packed sterilization material, in particular the polysaccharide, the condensation energy of the water is rapidly given off to the sterilization material. This leads to the heating up of the sterilization material and to the death of microorganisms optionally present in the aqueous dispersion or solution. On preferred operation with saturated steam, the medium acts as a heating medium and pressure-forming agent, which, as a counterpressure, equalizes the pressure generated in the packed aqueous dispersion by the temperature increase.

In a particularly preferred embodiment of the process according to the invention, the vaporizable medium is water, so the dissolution and the sterilization of the polysaccharide is effected by treatment of the packed aqueous dispersion with steam.

The process according to the invention is advantageously distinguished in that the heat treatment is carried out with the vaporized medium, preferably with steam, essentially in the absence of air and thus in a pure vapour atmosphere of the medium, preferably in a pure steam atmosphere.

According to the invention, provision can be made for the heat treatment to be performed during a period of 5 min to 30 min. Preferably, the heat treatment is carried out during a period of 20 min.

Preferably, the heat treatment is carried out at an elevated temperature, in particular between more than 100° C. and 140° C., in particular between 115° C. and 125° C., preferably at 121° C.

Preferably, the heat treatment is carried out at an elevated pressure of between 1 bar and 5 bar, in particular between 1 bar and 3 bar, preferably at 1 bar or 2 bar.

With advantage, standardized sterilization conditions are used for the sterilization of the aqueous polysaccharide dispersion. Preferably, the sterilization is carried out at a temperature of 121° C. and a pressure of 1 bar for 20 min. However, it may also be preferred to carry out the sterilization of the polysaccharide dispersion at a higher temperature for a shorter treatment period. For this purpose, a higher pressure is necessary. Such an organized standardized sterilization protocol provides for a temperature of 134° C. for 5 min at a pressure of 2 bar. While a sterilization temperature of 121° C. is unproblematical for the material of most packings, in particular of syringes, higher sterilization temperatures, in particular a sterilization temperature of 134° C., can lead to considerable problems with conventional packing materials. Such problems can be eliminated by the use of suitable materials, in particular plastics. Thus, as materials, in particular for syringes, cycloolefin copolymers, preferably of norbornene and ethylene, are used, which are marketed commercially, in particular under the name Topas® (Thermoplastic Olefin Polymer of Amorphous Structure).

According to the invention, provision can further be made for the heat treatment, preferably with steam, to be carried out in an autoclave, in particular in a sterilization autoclave. Such autoclaves advantageously have already standardized sterilization programmes, in particular at a temperature of 121° C. or 134° C.

The present invention moreover relates to a sterile polysaccharide solution of at least one polysaccharide in at least one packing, which is preferably completely filled with the solution. Preferably, it is a sterile polysaccharide solution of a polysaccharide in a packing which is preferably completely filled with the solution of a polysaccharide. In a particular embodiment of the solution according to the invention, it is a sterile dextran aldehyde solution.

In a further embodiment, the sterile polysaccharide solution has a higher concentration than corresponds to the solubility of the polysaccharide at room or surrounding temperature in water or a water/DMSO mixture. This is, as already mentioned, preferably achieved by introducing the desired amount of the polysaccharide into a specified volume of preferably prepared, in particular deionized, water. The suspension or paste or gel resulting therefrom is present after the sterilization process as a stable solution of the polysaccharide concerned. A stable solution in the sense of the present invention should be understood as meaning a solution which is not prone to subsequent deposition or precipitation of the polysaccharide concerned.

According to the invention, the sterile polysaccharide solution can have a content of 5% by weight to 20% by weight, in particular of 5% by weight to 15% by weight, preferably of 10% by weight, of polysaccharide.

In a further preferred embodiment, the sterile solution of the polysaccharide can be prepared as a gel by reaction with a second component, which is preferably likewise present in sterilized form. Preferably, the sterile polysaccharide solution can be prepared by reaction with a chitosan solution, in particular a 4% strength chitosan solution.

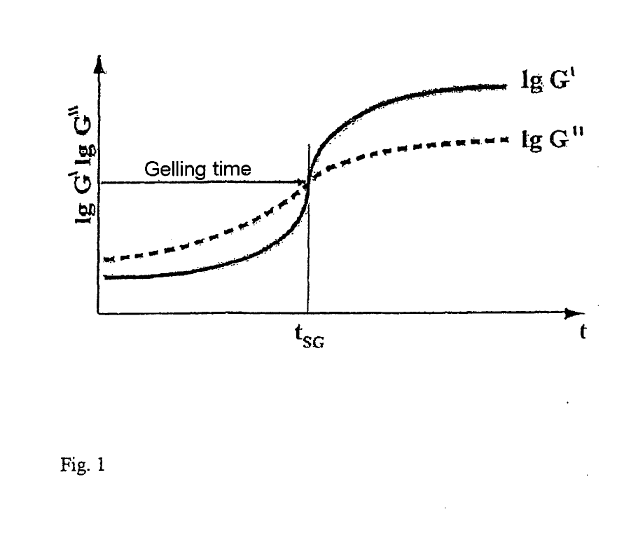

Preferably, the sterile polysaccharide solution can be prepared as a gel within 4 s to 30 s, in particular within 6 s to 9 s, preferably within about 7 s. The investigation of the gelling time is advantageously carried out using a rheometer. Owing to the gelling reaction commencing as a result of the mixing of the sterile polysaccharide solution and the chitosan solution, the elastic components in the gel being formed increase more rapidly than the viscous ones (gelling time corresponds to the point of intersection of the curves in FIG. 1).

Preferably, 60 s after the gelling time the gel has a loss factor δ of 3 to 10, in particular of 4 to 9, preferably of about 8, and 120 s after the gelling time has a loss factor δ of 2 to 6, in particular 3 to 5, preferably of about 4. The loss factor δ is a measure of the cohesive forces in the gel or of the strength of the gel, i.e. the higher the cohesive forces in the gel the stronger the gel. The smaller δ the greater the elastic components in the gel and thus also the cohesive forces. The loss factor results from the following equation:
tan δ=G″/G′
G″: viscous components
G′: elastic components

Advantageously, the packing of the polysaccharide solution is a packing of stable shape, in particular a syringe cylinder, for example a one-chamber or two-chamber syringe, which is contained in an in particular flexible, air-tight packing. As a flexible packing material, various materials are suitable, in particular plastic wrappings, which can be designed, for example, in the form of sachets. The packing for the polysaccharide solution is preferably already present, before sterilization, in a flexible packing material, so that the packing of the polysaccharide solution is also sterilized on its outside after sterilization.

According to the invention, it can be particularly advantageous for the syringe cylinder, in particular a one-chamber or two-chamber syringe, to be produced from a cycloolefin copolymer, in particular from norbornene and ethylene. Preferably, the syringe cylinder is produced from a material which is commercially obtainable under the name Topas®.

The advantage of these materials lies, as already mentioned, in their unproblematical handling at relatively high sterilization temperatures, in particular at a sterilization temperature of 134° C.

Finally, the invention also relates to all sterile polysaccharide solutions which are prepared or can be prepared by a process according to the invention.

The process according to the invention is distinguished compared with conventional sterilization processes in that the desired properties of the polysaccharides to be sterilized, in particular the physicochemical properties, are not affected or not adversely affected by the sterilization process. In comparison with conventional, in particular thermal, dissolving processes, the process according to the invention is also distinguished by a shorter dissolution time for polysaccharides which are poorly soluble in water, whereby, overall, shorter preparation times result for sterile polysaccharide solutions of polysaccharides which have a low water solubility. At the same time, the sterile polysaccharide solutions are provided by the process according to the invention in a ready-to-use form, preferably for medical and surgical application fields, which allows a simple and safe handling by the user, in particular the surgeon. A more labourious and expensive aseptic filling process, which necessarily follows in many conventional sterilization processes, in particular sterile filtration, and moreover means a not inconsiderable risk of contamination, in this way becomes superfluous.

Further features and details of the invention result from the following description of preferred embodiments in the form of examples. Here, the individual features can in each case be realized per se alone or multiply in combination with one another. The examples serve only to explain the present invention, which is to be in no way restricted thereto.

FIG. 1: Schematic representation of the time course of the viscous components (G″) and the elastic components (G′) during the gelling reaction between dextran aldehyde and chitosan. The point of intersection of the two curves corresponds to the gelling time.

### EXAMPLES

1. Preparation of an Aqueous Polymer Paste

A two generator pumping stages system and a generator are incorporated in a process pilot plant from IKA Werke (Staufen, Germany). A total of 2 l of pharmaceutical water (B/Braun, Melsungen Germany) is introduced. The introduction vessel and homogenizer are cooled to 6° C. 200 g of lyophilized dextran aldehyde are added continuously in a period of time of the 90 minutes. After addition is complete, the resulting aqueous polymer paste is homogenized for a further 30 minutes and filled into 5 ml one- and two-chamber syringes.

2. Steam Sterilization of an Aqueous Polymer Paste

The syringes are packed in TYVEK sachets inserted in a syringe holder and steam-sterilized in the autoclave. For this, a standard programme of 20 minutes at 121° C. is used. As a result of the sterilization, the pasty polymer gel is converted to a transparent liquid.

3. Proof of the Sterility

Spore strips which contained spores of the bacterium Geobacillus stearothermophilus having a content of 1×10⁶ spores were added before sterilization to in each case 20 one- and two-chamber syringes filled with dextran aldehyde paste (DA paste). The syringes were sterilized as described in Ex. 2 and subsequently checked for sterility. The tests were carried out by means of direct charging according to the requirements of Ph. Eur. 4 (2004). For this, the filters were incubated in Caso broth at 20-25° C. and in thioglycolate broth at 30-35° C. for 14 days. All syringes contained liquid and sterile dextran aldehyde solutions.

4. Preparation of a Supersaturated Dextran Aldehyde Solution (DA Solution), (Comparative Experiment)

100 g of dextran aldehyde are added to 1000 ml of pharmaceutical water and stirred at 60° C. After about 96 hours, the dextran aldehyde has completely dissolved. The clear solution was slowly cooled to room temperature.

5. Sterilization of the DA Solution (Comparative Experiment)

The completely dissolved DA solution from Ex. 4 is filled into one- or two-chamber syringes, packed in TYVEK sachets, inserted into a syringe holder and steam-sterilized in the autoclave. For this, a standard programme of 20 minutes at 121° C. is used.

6. Concentration of the Solutions

The water content of the lyophilized dextran aldehyde webs employed was determined by means of Karl Fischer titration in a quintuplicate determination. For the webs, a water content of 20% could be determined. The solutions prepared in examples 1 and 4 have a dextran aldehyde content of 7.8% (w/v). In a triplicate determination, the water content of the sterile solutions (Ex 2) and the paste (Ex. 1) was determined by gravimetric measurements.

| | | Sterile DA solution |
|---|---|---|
| | DA paste (Ex. 1) | (Ex. 2) |
| Content of | 7.5 ± 0.6% | 7.6 ± 0.3% |
| dextran aldehyde | | |
| [%] | | |

7. Determination of the Aldehyde Content

The influence of the sterilization on the chemical structure was carried out by determination of the aldehyde content. For this, the sterilized solutions of the paste were again lyophilized. The lyophilizates prepared were checked titrimetrically for the content of oxidized glucose units [B. T. Hofreiter, B. H. Alexander, I. A. Wolff, Anal. Chem. 1955, 27, 1930ff.].

0.15 g of dextran aldehyde (DA) is introduced into an Erlenmeyer flask and subsequently treated with 10 ml of a 0.25 N carbonate-free NaOH solution. The mixture is stirred until the dextran aldehyde employed has dissolved. The flask is then immersed for one minute in a hot water bath (80° C.) and subsequently placed in an ice bath with vigorous stirring. After one minute, 15 ml of 0.25 N sulphuric acid are added cautiously with stirring. The mixture is subsequently diluted with 50 ml of water and treated with 1 ml of 0.2% strength phenolphthalein solution. The acidic solution is titrated against the indicator using 0.25 N NaOH solution.

From the amounts of dextran or dextran aldehyde added, and the consumption of acid and base, the content of oxidized glucose units X is calculated as follows:

$$X = [ \frac{{( {n}_{eqBase} - {n}_{eqAcid} )}_{DA}}{\frac{{W}_{DA}}{161}} - \frac{( {n}_{eqBase} - {n}_{eqAcid} ) Dextran}{\frac{{W}_{Dextran}}{162}} ] \times 100 \%$$

X: Dialdehyde content
n_{eqAcid}: Equivalent substance amount of the acid
n_{eqBase}: Equivalent substance amount of the base
W_{DA}: Dry weight of dextran aldehyde
W_{Dextran}: Dry weight of dextran
n_{NaOH}: Normality of the NaOH titre

n_{H2SO4}: Normality of the H₂SO₄ solution used

| | Before preparation | |
|---|---|---|
| | of the paste | After sterilization |
| Content of oxidized | 96.6 ± 1.4 | 94.7 ± 0.9 |
| glucose units [%] | | |

Net result of Exs. 6 and 7: Chemical structure and concentration are not modified by the novel process.

8. Comparison of the Gelling Times with Chitosan Solutions

Dextran aldehyde solution and chitosan form a gel on mixing, which can be used as a surgical tissue adhesive, inter alia for haemostasis. In this connection, rapid gel formation (<10 s) is of crucial importance for the surgeon, in order that this adhesive is not rinsed from the wound area.

For the determination of the gelling time, 10 double-chamber syringes were filled with 5 ml total volume (Mixpac Systems, Red Cross Switzerland) in each case containing 1 ml of an aqueous 4% strength chitosan solution (Protasan® FMC Biopolymers, Drammen Norway) and 1 ml of polymer paste (Ex. 1). In parallel to this, 10 further double-chamber syringes were filled with 1 ml of a 4% strength chitosan solution and 1 ml of dextran aldehyde solution (Ex. 4). The syringes were subjected to steam sterilization, as described under Ex. 2 and Ex. 5.

The gelling time was investigated using a Gemini 150 rheometer (Malvern Instruments, Herrenberg Germany). Using a plate-plate measuring system, the time course of the gelling process was determined in oscillation, by placing a static mixer syringe (Mixpac Systems, Red Cross Switzerland) on the double-chamber syringe and injecting the mixture between the measuring plates.

| | | Sterile DA | |
|---|---|---|---|
| | | solution | |
| | Unsterile DA | prepared by | Sterile DA |
| | solution | sterilization | solution |
| | (Ex. 4) | of the paste | (Ex. 5) |
| | (prior art) | (Ex. 2) | (prior art) |
| Gelling time | 5.2 s ± 1.5 s | 7.0 s ± 1.3 s | 78.6 ± 26.7 s |
| with sterile | | | |
| 4% strength | | | |
| Protasan | | | |
| solution | | | |

9. Cohesive Forces of the Medical Gels:

The determination of the loss factor δ was likewise determined using the Gemini 150 rheometer.
tan δ=G″/G′

| | | Sterile DA | |
|---|---|---|---|
| | | solution | |
| | Unsterile DA | prepared by | Sterile DA |
| | solution | sterilization | solution |
| | (Ex. 4) | of the paste | (Ex. 5) |
| | (prior art) | (Ex. 2) | (prior art) |
| Loss factor δ | 13.2 ± 2.4 | 8.3 ± 1.1 | 28.0 ± 2.0 |
| 60 s after | | | |
| gelling time | | | |
| Loss factor δ | 6.9 ± 1.5 | 4.3 ± 1.6 | 14.1 ± 2.1 |
| 120 s after | | | |
| gelling time | | | |

Net result from Ex. 8 and 9:

The novel sterilization process of the DA paste leads to lower gelling times and markedly higher cohesive forces in comparison to the sterilization of the DA solution.

10. Time Between Preparing the Paste and Carrying Out the Sterilization

On standing for a relatively long time, the milky white paste changes its consistency to a transparent gel. The influence of the standing time on the properties of the resulting sterile solution was investigated. For this, a 2 l polymer paste (Ex. 1) was divided into 4 fractions, which were sterilized 1, 4, 7 or 9 days after paste preparation. The corresponding pastes were characterized according to the examples:

| Standing time | | |
|---|---|---|
| between paste | | |
| preparation and | | Content of |
| steam | Concentration % | oxidized glucose |
| sterilization | (w/v) (Ex. 6) | units (Ex. 7) |
| 1 day | 8.1 ± 0.5% | 93.7 ± 1.0% |
| 4 days | 7.3 ± 0.3% | 94.7 ± 0.9% |
| 7 days | 7.8 ± 0.8% | 93.2 ± 0.9% |
| 9 days | 7.8 ± 0.2% | 93.0 ± 0.5% |

| Standing time | Gelling time with | |
|---|---|---|
| between paste | 4% strength sterile | |
| preparation and | chitosan solution | Phase shift angle |
| steam sterilization | (Ex. 8) | δ after 60 s (Ex. 9) |
| 1 day | 7.2 ± 0.8 s | 4.2 ± 0.4 |
| 4 days | 7.5 ± 1.6 s | 3.9 ± 0.8 |
| 7 days | 8.8 ± 0.9 s | 3.5 ± 0.4 |
| 9 days | 9.2 ± 1.9% | 3.0 ± 0.3 |

Net Result: the standing time between paste preparation and steam sterilization has no influence on the properties of the resulting sterile dextran aldehyde solution.

11. Radiation Sterilization of the Polymer Paste:

Alternatively to steam sterilization, the polymer paste from Ex. 1 was also irradiated with E beam and gamma radiation. The doses used were 18 and 25 kGy. The polymer pastes were dissolved after the sterilization. Gelling with 4% strength chitosan solution took place, however, markedly more slowly. At the same time, the cohesive forces of the resulting solutions are markedly lower.

| Manner of | Gelling time with | |
|---|---|---|
| sterilization of | 4% strength sterile | |
| the 10% strength | chitosan solution | Phase shift angle |
| paste | (Ex. 8) | δ after 60 s (Ex. 9) |
| E beam 18 kGy | 13.4 ± 2.4 s | 20.1 ± 1.1 |
| E beam 25 kGy | 30.9 ± 4.7 s | 26.8 ± 1.9 |
| Gamma 18 kGy | 24.4 ± 4.2 s | 20.6 ± 1.3 |
| Gamma 25 kGy | 55.9 ± 15.3% | 35.3 ± 4.7 |

Net Result:

Steam sterilization is to be preferred to radiation sterilization.

1. Process for the preparation of sterile polysaccharide solutions, characterized in that at least one aqueous unsterile dispersion of at least one thermolabile and/or poorly soluble polysaccharide is packed and the dissolution of the polysaccharide and the sterilization are effected simultaneously by heat treatment of the packing at above 100° C.

2. Process according to claim 1, characterized in that for the heat treatment two polysaccharides present separately from one another, preferably reacting with one another, are used.

3. Process according to claim 1, characterized in that at least one modified polysaccharide, in particular bearing aldehyde and/or amino groups, is used.

4. Process according to claim 1, characterized in that at least one polysaccharide from the group consisting of hyaluronic acid, heparin, chitin, chitosan, alginate, cellulose, starch, amylase, amylopectin, dextran and its derivatives, of which at least one is poorly soluble, is used.

5. Process according to claim 1, characterized in that the polysaccharide used is dextran aldehyde.

6. Process according to claim 1, characterized in that the dispersion is prepared by introduction of the polysaccharide into water or into a water/DMSO mixture.

7. Process according to claim 6, characterized in that the introduction is performed during a time between 60 min and 120 min, in particular between 80 min and 100 min, preferably during 90 min.

8. Process according to claim 1, characterized in that the preparation of the dispersion is carried out at a temperature between 4° C. and 25° C., in particular between 4° C. and 15° C., preferably at 6° C.

9. Process according to claim 1, characterized in that for the preparation of the dispersion lyophilized polysaccharide is used.

10. Process according to claim 1, characterized in that the dispersion is homogenized.

11. Process according to claim 1, characterized in that the dispersion is prepared with a content of polysaccharide of 5 to 20% by weight, in particular 5 to 15% by weight, preferably of about 10% by weight.

12. Process according to claim 1, characterized in that the dispersion is packed in air-tight form.

13. Process according to claim 1, characterized in that the at least one dispersion is transferred to a closed container, preferably to a one- or two-chamber syringe.

14. Process according to claim 1, characterized in that the dispersion is sterilized in the form of a paste or of a gel.

15. Process according to claim 1, characterized in that the dissolution and sterilization is effected by treatment of the packed dispersion with steam.

16. Process according to claim 1, characterized in that the heat treatment is carried out with steam essentially in the absence of air, preferably in a pure steam atmosphere.

17. Process according to claim 1, characterized in that the heat treatment is performed for a period of 5 to 30 min, preferably for 20 min.

18. Process according to claim 1, characterized in that the heat treatment is carried out at an elevated temperature, in particular between more than 100° C. and 140° C., in particular between 115° C. and 125° C., preferably at 121° C.

19. Process according to claim 1, characterized in that the heat treatment is carried out at a pressure between 1 and 5 bar, in particular between 1 and 3 bar, preferably at 1 bar.

20. Process according to claim 1, characterized in that the heat treatment is carried out with steam in an autoclave.

21. Sterile polysaccharide solution of at least one thermolabile and/or poorly soluble polysaccharide in at least one packing, which is preferably completely filled with the solution.

22. Sterile polysaccharide solution according to claim 21, characterized in that it has a higher concentration of the polysaccharide than corresponds to its solubility at room temperature or surrounding temperature.

23. Sterile polysaccharide solution according to claim 21, characterized in that it has a content of 5 to 20% by weight, in particular 5 to 15% by weight, preferably of 10% by weight, of polysaccharide.

24. Sterile polysaccharide solution according to claim 21, characterized in that it can be prepared as a gel by reaction with a second component, in particular a chitosan solution.

25. Sterile polysaccharide solution according to claim 24, characterized in that the gel can be prepared within 4 s to 30 s, in particular within 6 s to 9 s, preferably within from about 7 s.

26. Sterile polysaccharide solution according to claim 24, characterized in that the gel after 60 s has a loss factor 5 of 3 to 10, in particular of 4 to 9, preferably of about 8, and after 120 s a loss factor 6 of 2 to 6, in particular 3 to 5, preferably of about 4.

27. Sterile polysaccharide solution according to claim 21, characterized in that the packing is a packing of stable shape, in particular a syringe cylinder, which is contained in an in particular flexible air-tight packing.

28. Sterile polysaccharide solution according to claim 27, characterized in that the syringe cylinder is produced from a cycloolefin copolymer, preferably from norbornene and ethylene.

29. Sterile polysaccharide solution, prepared by a process according to claim 1.

30. Sterile polysaccharide solution, which can be prepared by a process according to claim 1.
